(19) United States
(12) Patent Application Publication
(10) Pub. No.: US 2024/0269739 A1
(43) Pub. Date: Aug. 15, 2024

(54) NANOHYBRID AND NANOCOMPOSITE COMPOSITIONS AND METHODS FOR MAKING AND USING SAME

(71) Applicant: P&S Global Holdings LLC, Houston, TX (US)

(72) Inventors: Parash KALITA, Houston, TX (US); Jiang Wenping, Houston, TX (US); Rustom Mody, Houston, TX (US)

(73) Assignee: P&S Global Holdings LLC, Houston, TX (US)

(21) Appl. No.: 18/103,399

(22) Filed: Jan. 30, 2023

(51) Int. Cl. B22F 1/0545; B22F 1/103; B22F 1/145

(52) U.S. Cl. CPC B22F 1/0545; B22F 1/103; B22F 1/147; B82Y 5/00; B82Y 30/00; B82Y 40/00

(57) ABSTRACT

Disclosed herein are nanohybrid and nanocomposite compositions and methods. In a specific embodiment, the method for making a nanohybrid composition includes: contacting a carrier material with metallic salt precursors to make a first mixture, contacting the first mixture with a reducing agent reacting and a surface activated agent to make a second mixture, where the metallic salt precursors undergo chemical reduction to make metallic nanoparticles, and where the metallic nanoparticles are deposited onto the carrier material, contacting the second mixture with an organosilane coupling agent to make a nanohybrid composition.

### CROSS-REFERENCE TO RELATED APPLICATIONS

This patent application claims the benefit of and priority to U.S. Provisional Application No. 63/309,056 entitled, “Nanocomposites and Methods for Making and Using Same” filed on Feb. 11, 2022; U.S. Provisional Application No. 63/431,977 entitled, “Nanocomposites and Methods for Making and Using Same” filed on Dec. 12, 2022; PCT Application PCT/US21/032481, entitled “Nanohybrid Structures Containing Antimicrobial Nanoparticles” filed on May 14, 2021; and PCT/US21/43761, entitled “Microbial Water Purification and Sterilization Using Nanohybrid Structures Containing Antimicrobial Nanoparticles” filed Jul. 29, 2021, all of which are hereby incorporated by reference in their entirety.

### BACKGROUND

### Field

This application generally relates to nanohybrid and nanocomposite compositions. More specifically, this application relates to making and using the nanohybrid and nanocomposite compositions to inhibit the growth of contaminating microorganisms and their biofilms in a variety of commercial products.

### Description of Related Art

Microbe contamination of surfaces is a world-wide problem facing a number of industrial and public health sectors. For example, the attachment of pathogenic bacteria to titanium or other surgical implants can lead to serious post-operative complications, and may result in systemic infections. In another example, food-processing equipment must be maintained free of food-borne pathogens such as Escherichia coli and Salmonella typhimurium to avoid adverse health outcomes. The negative consequences of microbial surface colonization and contamination can be significant, causing substantial financial losses, severely impacting on human health and, in some cases, resulting in fatality.

The development of agents that are lethal to microbes or exhibit antimicrobial activity that reduce growth and propagation of microbes, is of commercial importance in a range of contexts. For example, the use of synthetic biocidal surfaces would have great utility and would contribute to efficiencies, improved health outcomes, reduced disease transmission and/or cost savings in areas such as health care provision and surgery, public health, sanitation and domestic hygiene, food processing, preparation, production and storage, animal husbandry, veterinary clinics, aged care facilities, schools and child care facilities, plumbing fixtures, waste treatment and recycling, amongst many others. Specifically, advantages can be envisaged in employing antimicrobial surfaces in fittings, devices and apparatus such as walls, floors, ceilings, hand rails, door knobs and handles, seat covers, tables, chairs, light switches, toilets, taps and other surfaces in public, commercial or domestic environments, food preparation surfaces, cooking and food preparation utensils and devices, food and beverage packaging and storage containers, food wrap, medical, surgical and dental tools, instruments and equipment, medical, dental and veterinary implants, hospital surfaces such as floors, walls, sinks, basins, bench tops, beds, mattress and pillow covers, hospital furniture, surgical, medical and food preparation gloves, hair dressing tools and equipment such as combs, brushes, razors and scissors, surfaces in commercial and domestic kitchens such as floors, walls, sinks, basins and bench tops, food and beverage mixers and processing/packaging devices or machines, food and beverage processing lines, abattoirs, protective clothing, goggles and glasses, water and pipes and tanks and fabric, textiles and clothing, especially protective clothing.

Currently, chemical antiseptics and disinfectants are used for the eradication of bacteria and other microbes. In the medical context antibiotics have protected countless lives since being discovered at the beginning of the last century. Besides directly curing infection related diseases, antibiotics have enabled the medical profession to undertake more sophisticated treatments with high risk of infection, such as organ transplantation and cancer chemotherapy [1]. However, the abuse of antibiotics has induced the rapid development of antibiotic-resistance, with the result that previously easily treatable diseases may again be deadly. The emergence of antibiotic resistance is a major global public health issue and challenge faced by healthcare systems [2], which is compounded by the highly adaptable nature of bacteria and their accelerated evolution brought about by over-prescription of antibiotics, their use in food production and by the processes of natural selection. A World Health Organization report highlighted that the current and foreseeable conventional antibiotic pipeline is insufficient to meet the rise in antibiotic resistance [3].

Consequently, there is a need new antimicrobial compositions that can be incorporated into plastics, fabrics, and filtration mediums.

### SUMMARY

Provided herein are nanohybrid and nanocomposite compositions and methods that have effective antimicrobial properties. In a specific embodiment, the method for making a nanohybrid composition includes: contacting a carrier material with metallic salt precursors to make a first mixture, contacting the first mixture with a reducing agent reacting and a surface activated agent to make a second mixture, where the metallic salt precursors undergo chemical reduction to make metallic nanoparticles, and where the metallic nanoparticles are deposited onto the carrier material, contacting the second mixture with an organosilane coupling agent to make a nanohybrid composition.

In another specific embodiment, the method for making a nanohybrid composition includes: contacting nanoparticles with a carrier material to make a first mixture, homogenizing the first mixture utilizing mechanochemical synthesis to make a homogeneous second mixture, and contacting the second mixture with an organosilane coupling agent to make a nanohybrid composition.

In yet another specific embodiment, the method for making a nanocomposite composition, wherein the method includes: contacting a non-metallic nanoparticle and a metal nanoparticle to make a nanocomposite particle, where the metallic nanoparticle is selected from the group comprising: silver, copper, zinc, selenium, and titanium, where non-metallic particles are selected from group consisting of: calcium carbonate and silicon dioxide, where the metallic nanoparticles have an average particle size in the range from 0.005 to 1 micron, and where the non-metallic particles have an average particle size in the range from 0.01 to 40 microns; and contacting the nanocomposite particle with a polymer to make a nanocomposite composition.

### BRIEF DESCRIPTION OF THE DRAWINGS

The present disclosure can be better understood by referring to the following drawings. The drawings constitute a part of this specification and include exemplary embodiments of the nanohybrid and nanocomposite compositions and methods, which can be embodied in various forms.

FIG. 1 is a graphical representation of the antimicrobial nanohybrid structures discussed herein, in which at least one of the size dimensions of the nanohybrid is ≤100 nm in at least one dimension.

FIG. 2 is a graphical representation of chemical bonding between organofunctionalized antimicrobial nanohybrid structures with organic polymers to form compatible organic/inorganic nanohybrids.

FIG. 3 presents example designs of microbial filtration unit with the antimicrobial nanohybrid media (by itself and in conjunction with other commercially available filter media).

FIG. 4 presents a summary of different polymer processing techniques that can be implemented for forming polymer nanohybrids with antimicrobial nanohybrid structures.

FIG. 5 presents an example of chemical reduction-based synthesis of nanohybrid structure and TEM image of the resultant nanohybrid structure.

FIG. 6 presents an example process sequence of organosilane treatment to form organofunctionalized Ag—ZnO—CaCO₃ nanohybrid for enhanced bonding with organic polymer composition.

FIG. 7 presents the growth study of acid producing bacteria (APB) in PRD vials inoculated with control (unfiltered) source water and water filtered through hydroxy-functional Ag-zeolite nanohybrid media (Test standard—NACE standard TM0194-2004).

FIG. 8 presents the Growth study of acid producing bacteria (APB) in inoculated with control (unfiltered) ground water and ground water filtered through hydroxy-functional Ag-Zeolite nanohybrid media (Test standard—APB-BART Protocol DBASOPO6).

FIG. 9 presents HAADF-STEM image of aminofunctional Ag—Cu—SiO₂ nanohybrid and EDX chemical analysis of the nanohybrid structure showing location and chemical identity of silver (Ag) and copper (Cu) nanoparticles.

FIG. 10 shows a method for depositing metallic nanoparticles on the surfaces of non-metallic particles to make a nanocomposite.

FIG. 11 shows a nanocomposite that include Ag nanoparticles (dark spots) deposited on calcium carbonate nanoparticles (light and gray).

FIG. 12 shows a photographic example of a standard disc used as a control in testing the efficacy of one embodiment of the claimed nanocomposite.

FIG. 13 shows a photographic example of a disc comprising polyethylene with A015.

FIG. 14 shows a graphical representation of the test results for the efficacy of reducing the growth of Staphylococcus aureus between standard disc and a disc made with A015.

FIG. 15 shows a graphical representation of the test results for the efficacy of reducing the growth of E. coli between standard disc and a disc made with A015.

### DETAILED DESCRIPTION

In one or more embodiments, the nanohybrid compositions can include, but are not limited to: one or more substrates, one or more nanostructures, one or more nanohybrid structures, one or more nanocomposites, one or more nanoparticles, one or more metallic particles, one or more non-metallic particles, one or more metals, one or more metal oxides, one or more metallic salt precursors, one or more reducing agents, one or more coatings, one or more layers, one or more microparticles, one or more carrier materials, one or more matrix materials, one or more zeolites, one or more polymers, one or more resins, one or more surface active agents, one or more organosilanes, and one or more additives. In one or more embodiments, the nanocomposite compositions can include, but are not limited to: one or more substrates, one or more nanostructures, one or more nanoparticles, one or more metallic particles, one or more non-metallic particles, one or more microparticles, one or more metals, one or more metallic salt precursors, one or more reducing agents, one or more coatings, one or more layers, one or more polymers, one or more resins, one or more zeolites, and one or more additives.

The one or more nanoparticles can be chemically and/or mechanically deposited on to a substrate to form a hybrid structure and/or a layered structure. In another example, the nanohybrid structures can be made by nanoscale modifications of organic/inorganic materials with antimicrobial nanoparticles and organofunctional reactive groups for better distribution of antimicrobial species, stronger adsorptive behavior, and wettability. Such a nanohybrid structures can provide enhanced antimicrobial surface contact when water is treated with or permeated through the nanohybrid structures for microbial purification and decontamination (disinfection and sterilization) purposes.

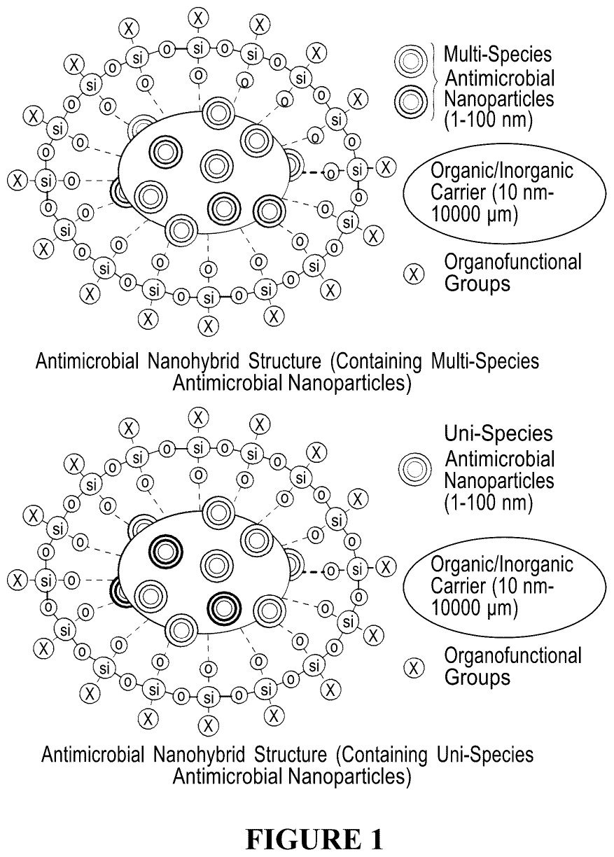

In an embodiment, the nanohybrid compositions (as shown in FIG. 1) can include from about 0.5 wt % to about 50 wt % of inorganic nanoparticles that are deposited onto the one more carrier materials e, and the entire nanoparticles-deposited inorganic/organic structure is organofunctionalized with organofunctional groups, wherein the inorganic antimicrobial nanoparticles (particles with at least one dimension≤100 nm). In another example, the one or more organosilanes can be used for organofunctionalization of the inorganic and/or hybrid compositions to form organic-inorganic nanohybrid structures.

The one or more metallic nanoparticles can include: silver, copper, zinc, selenium, titanium, and mixtures thereof. In another embodiment, the non-metallic particles can include, but are not limited to: calcium carbonate, silicon dioxide, and mixtures thereof.

The one or more nanoparticles can include, but are not limited to: silver, silver oxide, one or more silver compounds, copper, copper oxide, one or more copper compounds, zinc, zinc oxide, nickel, nickel oxide, one or more nickel compounds, selenium, selenium oxide, one or more selenium compounds, titanium, titanium dioxide, one or more titanium compounds, and mixtures thereof. The copper, silver, zinc, and titanium nanoparticles can be made by reducing copper-based salts silver-based salts silver-based salts, and titanium-based salts, respectively. The one or more metallic salt precursors can include, but are not limited to: any hydrolyzable or water-soluble metallic salts which can be reduced to metal or metal oxide nanoparticles. For example, copper/copper oxide nanoparticles can be derived by reducing copper (II) salts.

The copper nanoparticles can include, but are not limited to: copper sulfate (Cu₂SO₄), copper chloride (CuCl₂), copper hydroxide (Cu(OH)₂), copper nitrate (Cu(NO₃)₂, copper fluoride (CuF₂), copper acetate (Cu(OAc)₂), copper bromide (CuBr₂), copper formate (C₂H₂CuO₄), copper phosphate (Cu₃(PO₄)₂·n(H₂O)), copper chromite (Cu₂Cr₂O₅), copper hexafluorosilicate (CuF₆Si), copper selenate (CuO₄Se), and mixtures thereof.

The silver nanoparticles can include, but are not limited to: silver nitrate (AgNO₃), silver fluoride (AgF₂), silver nitrite (AgNO₂), silver perchlorate (AgClO₄), silver carbonate (AgCO₃), silver chloride (AgCl₂), and mixtures thereof.

The zinc nanoparticles can include, but are not limited to: zinc oxide (ZnO), zinc chloride, zinc nitrate, zinc acetate, and mixtures thereof.

The titanium nanoparticles can include, but are not limited: titanium oxide, titanium tetra-isopropoxide (TTIP), titanium tetrachloride (TiCl₄), metatitanic acid [TIO(OH)₂] and titanium oxide sulphate (TiOSO₄). titanium oxide, titanium tetra-isopropoxide (TTIP), titanium tetrachloride (TiCl₄), metatitanic acid [TiO(OH)₂], titanium oxide sulphate (TiOSO₄), and mixtures thereof.

In an embodiment, the nanocomposites can have more surface area than nanocomposites that do not include metallic nanoparticles. The nanocomposites can release metallic ions, resulting in stronger activity with less metallic content. The metal-based nanoparticles that has inherent antimicrobial property to kill or stop the growth microorganisms (bacteria, fungi, and/or viruses). The antimicrobial properties of the above metal nanoparticles are known in the art [4].

The one or more nanoparticles can include size dimensions that vary widely. For example, the one or more metallic nanoparticles can have an average particle size that varies widely. For example, the one or more metallic nanoparticles can have an average particle size from a small of about 0.005 microns, about 0.01 microns, or about 1.0 micron, to a high of about 10.0 microns, about 20.0 microns, or about 30.0 microns. For example, the one or more metallic nanoparticles can have an average particle size from a small of about 0.005 microns to about 0.01 microns, about 0.05 microns to about 1.0 microns, about 0.1 microns to about 1.5 microns, about 1.0 microns to about 10.0 microns, about 5.0 microns to about 1.0 microns, or about 15 microns to about 45 microns.

In another embodiment, the non-metallic particles can include, but are not limited to: calcium carbonate, silicon dioxide, zinc oxide, titanium oxide, magnesium oxide, aluminum oxide, one or more silica sands, one or more zeolites, and mixtures thereof. The one or more zeolites can include, but are not limited to: one or more analcimes, one or more chabazites, one or more clinoptilolites, one or more erionite, one or more ferrierite, one or more heulandites, one or more laumontites, one or more mordenites, one or more phillipsites, and mixtures thereof.

The one or more non-metallic particles can include size dimensions that vary widely. For example, the one or more non-metallic particles can have an average particle size that varies widely. The one or more metallic nanoparticles can have an average particle size from a small of about 0.01 microns, about 1.0 microns, or about 10.0 micron, to a high of about 40.0 microns, about 50.0 microns, or about 80.0 microns. For example, the one or more metallic nanoparticles can have an average particle size from a small of about 0.01 microns to about 50.0 microns, about 0.1 microns to about 10.0 microns, about 1.0 microns to about 25.0 microns, about 20.0 microns to about 50.0 microns, about 35.0 microns to about 79.0 microns, or about 50 microns to about 65 microns.

The one or more nanoparticles can include size dimensions that vary widely. For example, the nanoparticles can include a length from a short of about 1 nm, about 5 nm, or about 50 nm, to a long of about 100 μm, about 500 μm, or about 1,000 nm. In another example, the nanoparticles can include a length from about 1 nm to about 1,000 nm, about 2 nm to about 10 μm, about 5 nm to about 20 nm, about 10 nm to about 100 nm, about 50 nm to about 250 nm, about 100 nm to about 500 nm, or about 250 nm to about 750 nm. In another example, the nanoparticles can include a radius from a short of about 10 nm, about 20 nm, or about 50 nm, to a long of about 1,000 nm, about 5,000 nm, or about 10,000 nm. In another example, the nanoparticles can include a radius from a short of about 1 nm, about 5 nm, or about 50 nm, to a long of about 100 μm, about 500 μm, or about 1,000 nm. In another example, the nanoparticles can include a radius from about 1 nm to about 1,000 nm, about 2 nm to about 10 μm, about 5 nm to about 20 nm, about 10 nm to about 100 nm, about 50 nm to about 250 nm, about 100 nm to about 500 nm, or about 250 nm to about 750 nm. In another example, the nanoparticles can include a radius from a short of about 10 nm, about 20 nm, or about 50 nm, to a long of about 1,000 nm, about 5,000 nm, or about 10,000 nm.

The nanoparticles can include a surface area that varies widely. For example, the nanoparticles can include surface area can be from a low of about 2.5 m²/g, about 4.0 m²/g, or about 7.0 m²/g to a high of about 25.0 m²/g, about 55.0 m²/g, or about 65.0 m²/g. In another example, the nanoparticles can include surface area can be from about 2.5 m²/g to about 65.0 m²/g, about 4.5 m²/g to about 10.0 m²/g, about 8.5 m²/g to about 35.0 m²/g, or about 25.0 m²/g to about 55.0 m²/g.

The content of the one or more nanoparticles in the nanohybrid compositions can vary widely. For example, the content of the inorganic nanoparticles in the nanohybrid compositions can be from a low of about 0.005 wt %, about 0.05 wt %, or about 1.0 wt %, to a high of about 30.0 wt %, about 40.0 wt %, or about 50.0 wt %. In another example, the content of the inorganic nanoparticles in the nanohybrid compositions can be from about 0.005 wt % to about 50.0 wt %, about 1.0 wt % to about 45.0 wt %, about 0.06 wt % to about 5.0 wt %, about 0.7 wt % to about 2.7 wt %, about 2.5 wt % to about 40.0 wt %, about 5.0 wt % to about 35.0 wt %, about 1.5 wt % to about 48.0 wt %, or about 10.0 wt % to about 20.0 wt %. The weight percent of the one or more nanoparticles in the nanohybrid compositions can be based on the total weight of the nanohybrid compositions composition.

The content of the one or more nanoparticles in the nanocomposite compositions can vary widely. For example, the content of the nanoparticles in the nanocomposite compositions can be from a low of about 0.005 wt %, about 0.05 wt %, or about 1.0 wt %, to a high of about 30.0 wt %, about 40.0 wt %, or about 50.0 wt %. In another example, the content of the nanoparticles in the nanocomposite compositions can be from about 0.005 wt % to about 50.0 wt %, about 0.05 wt % to about 45.0 wt %, about 0.006 wt % to about 5.0 wt %, about 0.07 wt % to about 2.7 wt %, about 2.5 wt % to about 40.0 wt %, about 5.0 wt % to about 35.0 wt %, about 1.5 wt % to about 48.0 wt %, or about 10.0 wt % to about 20.0 wt %. The weight percent of the one or more nanoparticles in the nanocomposite compositions can be based on the total weight of the nanocomposite compositions.

The one or more carrier materials and/or matrix materials can include, but not limited to: one or more inorganic carrier materials, one or more organic carrier materials, and mixtures thereof. The organic carrier materials and/or matrix material can include, but are not limited to: one or more chitosan, one or more starch, one or more lignin, one or more nanocrystalline, one or more nano-fibrillated cellulose, one or more thermoplastic organic polymers, one or more thermosets organic polymers, and mixtures thereof. The inorganic carrier materials and/or matrix material can include, but are not limited to: one or more zeolites, one or more granular clinoptilolite zeolites, one or more chabazite, one or more activated carbons, one or more activated alumina, manganese dioxide, one or more anthracites, one or more BIRM® by Clark Corporation, calcite, magnesium oxide, one or more silica sands, one or more diatomite, zinc oxide, titanium dioxide, one or more graphenes, one or more graphene oxides, one or more garnets, and mixtures thereof.

The nanohybrids compositions and/or the nanocomposite compositions can include thermoplastic polymers, thermosetting polymers, and biopolymers as continuous phase or matrix, and the nanohybrid(s) incorporation/reinforcement process can be accomplished in solid, semi-solid, and liquid phase of matrix polymers. The one or more polymers can include, but are not limited to: one or more polypropylenes, one or more polyurethanes, one or more polyesters, one or more polystyrenes, one or more cellulose acetates, one or more polyvinylidene fluorides, one or more polyvinyl chlorides (PVC), one or more polysulfones, one or more polyacrylonitriles, one or more polyethersulfones, one or more polyethylene glycols (PEG), one or more polybutylene terephthalate (PBT), one or more polyvinyl alcohols (PVA), one or more poly(methyl methacrylates) (PMMA), one or more polyacryletherketones (PAEK), one or more polyethylenimines (PEI), one or more polyanilines, one or more polyurethane, one or more aliphatic polyamide, one or more aromatic polyamides, one or more polyethersulfone amides, one or more styrene acrylonitriles, one or more polyether ether ketones (PEEK), one or more polypropylenes (PP) and its variants, one or more polyvinyl chlorides (PVC), one or more nylons, one or more low-density polyethylenes (LDPE) and its variants, one or more linear low-density polyethylenes (LLDPE) and its variants, one or more high-density polyethylenes (HDPE) and its variants, one or more polyutheranes (PU) and its variants, one or more polyacrylics, one or more polyamides, one or more nylons (6, 66, 6/6-6, 6/9, 6/10, 6/12, 11 & 12), one or more polycarbonate, one or more polystyrene, one or more acrylonitrile butadiene styrene (ABS), one or more polyvinyl chloride (PVC), one or more TEFLON® by E.I. Du Pont de Nemours and Company, one or more polyesters, one or more polyacrylic acids (PAA), one or more epoxys, one or more phenolic polymers, one or more phenol formaldhydes, one or more vinyl esters, one or more polyurethanes, one or more fluoropolymers, one or more cyanate esters, one or more polyesters, one or more urea formaldehydes, one or more silicones, one or more polysiloxanes, and mixtures thereof. The one or more biopolymers can include, but are not limited to: one or more isoprene polymers, one or more natural polyphenolic polymers, one or more cellulose, one or more nanocelluloses, one or more lignins, one or more melanins, one or more complex polymers of long-chain fatty acids, and mixtures thereof. The organofunctionalized nanohybrid compositions can provide effective chemical bridging and bonding for enhanced compatibility, adhesion, self-assembly, and spatial distribution within the polymer matrix for rendering antimicrobial activity and durability. In addition, the organofunctionalized nanohybrid composition can provide decreased critical surface tensions with hydrophilic/polar silane treatment, which can enhance the adsorptive behavior of the nanohybrid composition to increase surface area interaction of water with the antimicrobial nanoparticles for superior inhibition of deleterious microbes.

The one or more zeolites can include, but are not limited to: one or more analcimes, one or more chabazites, one or more clinoptilolites, one or more erionite, one or more ferrierite, one or more heulandites, one or more laumontites, one or more mordenites, one or more phillipsites, and mixtures thereof.

The one or more carrier materials and/or matrix materials can be surface functionalized with the one or more organosilane coupling agents so that the nanohybrid structures contains reactive organofunctional groups to catalyze further reactions and impart hydrophilic or hydrophobic surface behavior. The organosilanes can include a reactive organofunctional group (represented as X) and three hydrolyzable groups (represented as Y), as shown below:

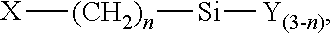

where n is an integer from 1 to 2.

The one or more surface active agents can include, but are not limited to: cyclodextrin, poly(vinyl pyrrolidone), poly(ethylene glycol), poly(vinyl alcohol), sodium dodecyl benzenesulfonate, abietic acid, polyethoxylated octyl phenol, sorbitan monoester, glycerol diester, dodecyl betaine, N-dodecyl pyridinium chloride, sulfosuccinate, 2-bis(ethyl-hexyl) sodium sulfosuccinate, alkyl dimethyl benzyl-ammonium chloride, cetyl trimethyl ammonium bromide, and hexadecyl trimethyl ammonium bromide; preferred surface active agents are cyclodextrin, poly(ethylene glycol), poly(vinyl pyrrolidone), poly(vinyl alcohol), sorbitan monoester, glycol diester; and the most preferred are poly(ethylene glycol) and poly(vinyl pyrrolidone). During chemical reduction, a water and alcohol weight ratio can include not more than 70 wt % of the alcohol in the aqueous mixture.

The one or more surface active agents can be any surfactant or dispersant containing cationic, anionic, non-ionic, and zwitterionic groups or the combination of any two of the functional groups in one molecule. The surface active agents can include, but are not limited to: cyclodextrin, poly(vinyl pyrrolidone), poly(ethylene glycol), poly(vinyl alcohol), sodium dodecyl benzenesulfonate, abietic acid, polyethoxylated octyl phenol, sorbitan monoester, glycerol diester, dodecyl betaine, N-dodecyl pyridinium chloride, sulfosuccinate, 2-bis(ethyl-hexyl) sodium sulfosuccinate, alkyl dimethyl benzyl-ammonium chloride, cetyl trimethyl ammonium bromide, and hexadecyl trimethyl ammonium bromide; preferred surface active agents are cyclodextrin, poly(ethylene glycol), poly(vinyl pyrrolidone), poly(vinyl alcohol), sorbitan monoester, glycol diester; and the most preferred are poly(ethylene glycol) and poly(vinyl pyrrolidone). During chemical reduction, water and alcohol weight ratio can include not more than 70 wt % of the alcohol in the aqueous mixture.

The one or more silane coupling agents can include, but are not limited to: glycol functional group-3-[methoxy(polyethyleneoxy)6-9]propyltrimethoxysilane; methoxy PEG silane; amino functional group-3-aminopropyltrimethoxysilane; 3-aminopropyltriethoxysilane; 2-dimethoxy-1,6-diaza-2-silacyclooctane; N-(2-aminoethyl)-2,2,4-trimethyl-1-aza-2-silacyclopentane; N-(3-aminopropyldimethylsilyl)aza-2,2-dimethyl-2-silacyclopentane; mercapto functional group-3-mercaptopropyltrimethoxysilane; 3-mercaptopropylmethyldimethoxysilane; epoxy functional group-2-(3,4 epoxycyclohexyl) ethyltrimethoxysilane; 3-glycidoxypropyl trimethoxysilane; 3-glycidoxypropyl methyldiethoxysilane; methacryloxy group-3-methacryloxypropyl triethoxysilane; 3-methacryloxypropyl methyldimethoxysilane; 3-methacryloxypropyl methyldiethoxysilane; isocyanate functional group-3-isocyanatopropyltriethoxysilane; acryloxy functional group-3-acryloxypropyl trimethoxysilane; ureide group-3-ureidopropyltrialkoxysilane; and other silanes can include, but are not limited to: (3-methacrylamidopropyl)triethoxysilane; triethoxysilylpropoxy(polyethyleneoxy) dodecanoate; N-(3-triethoxysilylpropyl) gluconamide; N-(triethoxysilylpropyl)-O-polyethylene oxide urethane; (2-diethylphosphatoethyl) methyldiethoxysilane; 3-(N-acetyl-4-hydroxyprolyloxy)propyltriethoxysilane; bis[(3-methyldimethoxysilyl)propyl]polypropylene oxide; and mixtures thereof. They can be selected from the following based on criteria of physical dimension of the substrates, number, and type of surface hydroxyl groups on the substrates (substrates in this case are the nanohybrid structures), and surface properties. The one or more silane coupling agents can be used for the organofunctional modification of nanohybrid structure.

The content of the one or more carrier materials and/or matrix material in the nanohybrid compositions can vary widely. For example, the content of the carrier materials in the nanohybrid compositions can be from a low of about 0.5 wt %, about 1.0 wt %, or about 2.0 wt %, to a high of about 30.0 wt %, about 50.0 wt %, or about 99.9 wt %. In another example, the content of the carrier materials in the nanohybrid compositions can be from about 0.5 wt % to about 50.0 wt %, about 1.0 wt % to about 45.0 wt %, about 0.6 wt % to about 5.0 wt %, about 0.7 wt % to about 2.7 wt %, about 2.5 wt % to about 40.0 wt %, about 5.0 wt % to about 35.0 wt %, about 1.5 wt % to about 48.0 wt %, about 10.0 wt % to about 20.0 wt % about 20.0 wt % to about 99.0 wt %, about 21.0 wt % to about 32.0 wt %, about 25.0 wt % to about 50.0 wt %, about 30.0 wt % to about 79.0 wt %, about 40.0 wt % to about 89.0 wt %, and about 55.0 wt % to about 95.0 wt %. The weight percent of the one or more carrier materials and/or matrix material can be based on the total weight of the nanohybrid compositions composition.

The content of the one or more carrier materials and/or matrix material in the nanocomposite compositions can vary widely. For example, the content of the carrier materials in the nanocomposite compositions can be from a low of about 0.5 wt %, about 1.0 wt %, or about 2.0 wt %, to a high of about 30.0 wt %, about 50.0 wt %, or about 99.9 wt %. In another example, the content of the carrier materials in the nanocomposite compositions can be from about 0.5 wt % to about 50.0 wt %, about 1.0 wt % to about 45.0 wt %, about 0.6 wt % to about 5.0 wt %, about 0.7 wt % to about 2.7 wt %, about 2.5 wt % to about 40.0 wt %, about 5.0 wt % to about 35.0 wt %, about 1.5 wt % to about 48.0 wt %, about 10.0 wt % to about 20.0 wt % about 20.0 wt % to about 99.0 wt %, about 21.0 wt % to about 32.0 wt %, about 25.0 wt % to about 50.0 wt %, about 30.0 wt % to about 79.0 wt %, about 40.0 wt % to about 89.0 wt %, and about 55.0 wt % to about 95.0 wt %. The weight percent of the one or more carrier materials and/or matrix material can be based on total weight of the nanocomposite composition.

The content of the one or more polymers in the nanohybrid compositions and/or nanocomposite compositions can vary widely. For example, the content of the polymers in the nanohybrid compositions and/or nanocomposite compositions can be from a low of about 20.0 wt %, about 30.0 wt %, or about 40.0 wt %, to a high of about 80.0 wt %, about 90.0 wt %, or about 99.0 wt %. In another example, the content of the polymers in the nanohybrid compositions and/or nanocomposite compositions can be from about 20.0 wt % to about 99.0 wt %, about 21.0 wt % to about 32.0 wt %, about 25.0 wt % to about 50.0 wt %, about 30.0 wt % to about 79.0 wt %, about 40.0 wt % to about 89.0 wt %, and about 55.0 wt % to about 95.0 wt %. The weight percent of the polymers can be based on the total weight of the nanohybrid compositions composition, or based on the total weight of the nanocomposite composition.

The carrier material can include size dimensions that vary widely. For example, the carrier material can include a length from a short of about 10 nm, about 20 nm, or about 50 nm, to a long of about 1,000 μm, about 5,000 μm, or about 10,000 μm. In another example, the carrier material can include a length from about 10 nm to about 10,000 μm, about 20 nm to about 9,000 μm, about 50 nm to about 1,000 μm, about 10 nm to about 10,000 μm, about 10 nm to about 1,000 nm, about 10 nm to about 10,000 μm, about 10 nm to about 10,000 μm, or about 10 nm to about 10,000 μm. In another example, the carrier material can include a radius from a short of about 10 nm, about 20 nm, or about 50 nm, to a long of about 1,000 μm, about 5,000 μm, or about 10,000 μm. In another example, the carrier material can include a radius from about 10 nm to about 10,000 μm, about 20 nm to about 9,000 μm, about 50 nm to about 1,000 μm, about 10 nm to about 10,000 μm, about 10 nm to about 1,000 nm, about 10 nm to about 10,000 μm, about 10 nm to about 10,000 μm, or about 10 nm to about 10,000 μm.

The one or more reducing agents can include, but are not limited to: citric acid, boric acid, hydrazine monohydrate, butyl aldehyde, diethylene glycolmonobutyl ether, sodium boric acid, sodium citrate, ascorbic acidcetyltrimethyl ammonium bromide, ammonia, sodium hydroxide, hydrogen peroxide, hydroxyl benzaldehyde, and mixtures thereof. The reducing agents can include acidic compounds, basic compounds, or PH neutral compounds.

The one or more additives can include, but are not limited to: one or more surfactants, one or more coloring pigments, one or more reinforcing materials, one or more antioxidants, one or more UV stabilizer, one or more plasticizers, one or more antistatic agents, and mixtures thereof. The compounded polymer-antimicrobial nanohybrid blend can be fed directly or can be converted into solid pellets, composite resins, and blends before feeding to the shaping/forming processes.

The content or concentration of the one or more additives in the nanohybrid compositions can vary widely. For example, the nanohybrid compositions and/or nanocomposite compositions can have a concentration of the one or more additives from a low of about 0.1 wt %, about 1.0 wt %, or about 5.0 wt %, to a high of about 10.0 wt %, about 40.0 wt %, or about 50.0 wt %. In another example, the nanohybrid compositions and/or nanocomposite compositions can have a concentration of the one or more additives from about 0.1 wt % to about 50.0 wt %, about 1.0 wt % to about 10.0 wt %, about 2.0 wt % to about 25.0 wt %, or about 3.0 wt % to about 30.0 wt %. The weight percent of the one or more additives can be based on the total weight of the nanohybrid compositions composition, or based on the total weight of the nanocomposite composition.

In one or more embodiments, the methods to make nanohybrid composition can include, but are not limited to: manufacturing of inorganic antimicrobial nanoparticles deposited to organic/inorganic carrier materials and organofunctionalization of compound derived from the first step. The first step can be accomplished through two different processes, (a) chemical reduction, or (b) mechanochemical synthesis. The selection of these two processes depends on the desired inorganic-organic hybrid composition and physical dimensions of inorganic antimicrobial nanoparticles and organic/inorganic carrier materials.

The mechanical milling or chemical reduction is carried out in a matrix of carrier materials to deposit the as-produced antimicrobial nanoparticles on the carrier materials.

The nanoparticles (e.g., metal and/or metal oxides) can be derived either by mechanochemical synthesis of milling large sized particles into nanosized particles (for example, milling 2.5 μm ZnO particles to less than 100 nm ZnO nanoparticles) or by chemical reduction of metallic salt precursors into metal/metal oxide nanoparticles (e.g., reducing silver nitrate salt to silver nanoparticles or copper sulphate salt to copper oxide nanoparticles).

The one or more mechanochemical steps in the synthesis can include, but are not limited to: high-energy mechanical/ball milling is a nanomanufacturing method in which mechanical and chemical phenomena are coupled on a molecular scale to from nanosized particles and as well as composite and/or hybrid particles with uniform grain sizes and complex compositions. Mechanical milling or chemical reduction is carried out in a matrix of inorganic carrier materials to deposit the as-produced antimicrobial nanoparticles onto the inorganic carrier materials.

Mechanical milling or chemical reduction is carried out in a matrix of inorganic carrier materials to deposit the as-produced antimicrobial nanoparticles onto the inorganic carrier materials. For example, the mechanochemical synthesis can include placing the inorganic antimicrobial particles (e.g., silver and/or zinc oxide) and the organic/inorganic carrier materials (e.g., zeolite, chitosan, and other carrier materials), and optionally additives, in an appropriate size ratios and concentrations to a high-energy mill (attritor or ball mill) loaded with milling media (ceramic or hardened steel balls). The reactants can be ball milled for specific periods to produce structures with desired compositional and morphological characteristics. The expanded movement of media at high RPMs exerts various forces such as impact, rotational, shear, and tumbling leading to repeated fracturing, cold welding, amorphization, and rewelding of blended particles to yield a homogeneous compound from dissimilar materials (e.g., a composition of silver-ZnO-zeolite) and at the same time, size reductions and shape modifications as a function of milling time and ratio of milling media to reactants. For manufacturing the nanohybrid compositions, the mechanochemical synthesis can be performed in two ways: direct milling/grinding involving only the reactants (antimicrobial and carrier materials) and other in the presence of auxiliary additives (usually liquids and/or ions) with the reactants. The later can significantly increase the activity of the reactants for thorough and easy reactions. The auxiliary additives can be selected from but not limited to water (H₂O), salts (sodium chloride, potassium dichromate, potassium nitrate, copper sulphate and alkali metal salts) and/or organic solvents (methanol, ethanol, propylene glycol, propanol, cyclohexane, benzene, toluene, cyclohexanone, ethers, and chlorinated solvents). Examples of organic solvents are listed in Joshi et al [5], which is incorporated herein as reference. Following mechanochemical synthesis, the nanohybrid composition becomes surface functionalized by combining it with appropriate organosilane coupling agents. The Mechanical milling or chemical reduction can be carried out in a matrix of inorganic carrier materials to deposit the as-produced antimicrobial nanoparticles onto the inorganic carrier materials.

In an embodiment, the chemical reduction reaction can include the carrier materials being contacted with a solution containing one or more metallic salt precursors to make a mixture (selected for desired antimicrobial nanoparticles, e.g., silver nitrate salt for silver nanoparticles, copper chloride salt for copper nanoparticles, and the like). A reducing agent and/or a surface-activated agents can be added to the mixture, either with or without the presence of short-chain organic alcohols at 55-135° C. under positive pressure. The reaction mixture is then dry cured under vacuum between 60-120° C. followed by dry milling into fine powder particulates. The reduction reaction followed by thermal processing can yield consistent-sized antimicrobial nanoparticles (10-100 nm) deposited on the inorganic/organic material matrix. Next, the nanohybrid composition becomes surface functionalized by combining it with appropriate organosilane coupling agents.

In an embodiment, the nanohybrid composition can include a polymer and/or copolymer as a continuous phase and/or matrix containing dis-continuous and/or dispersed phase of the nanohybrid structures. The nanohybrid structures can be combined with polymers by incorporating, bonding, and reinforcing polymers with the organofunctionalized nanohybrids structures containing antimicrobial nanoparticles to make the nanohybrid compositions. Incorporation of nanohybrids (e.g., aminofunctionalized Ag—ZnO nanohybrid) can impart antimicrobial characteristics to the polymer composition and end-use membranes and filters manufactured from such polymers.

The nanocomposite, in weight percentage of about 0.1 wt % to about 50.0 wt %, can be added to one or more with polymers at elevated temperature. The compound is then extruded as wires and chopped as pellets of different sizes. The pellets can be used as additives to the same polymers and mixed as a composite for other processing such as rotary molding, injection molding, blow molding, and extrusion. In such a composite structure, it is expected to provide long-lasting anti-microbial, anti-odor, and anti-stain functions. Specifically, the inventive nanocomposite powder is included in polymeric structure of water storage tanks and its accessories including pipes, filters, connectors, etc.

The reaction of nanohybrids structures with organosilanes (e.g., aminofunctional silane) involves four steps that can occur simultaneously. An embodiment of a reaction scheme for the surface functionalized with organosilane coupling agents is shown below:

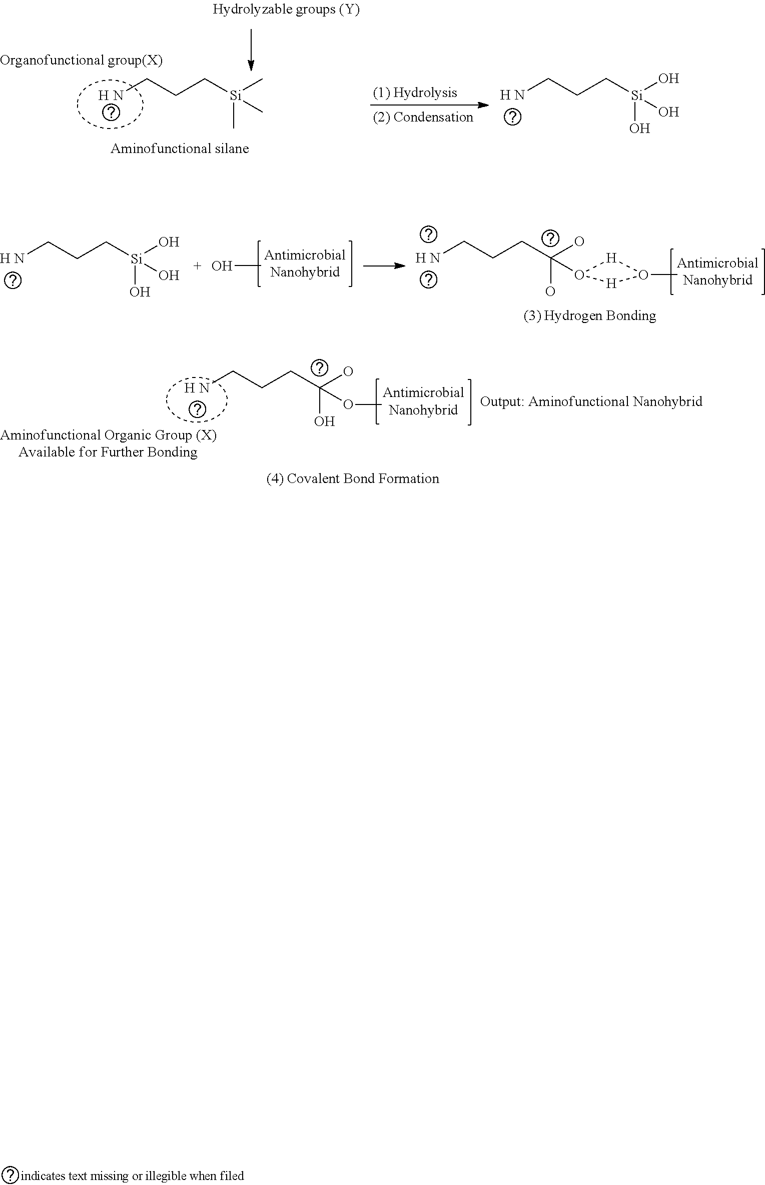

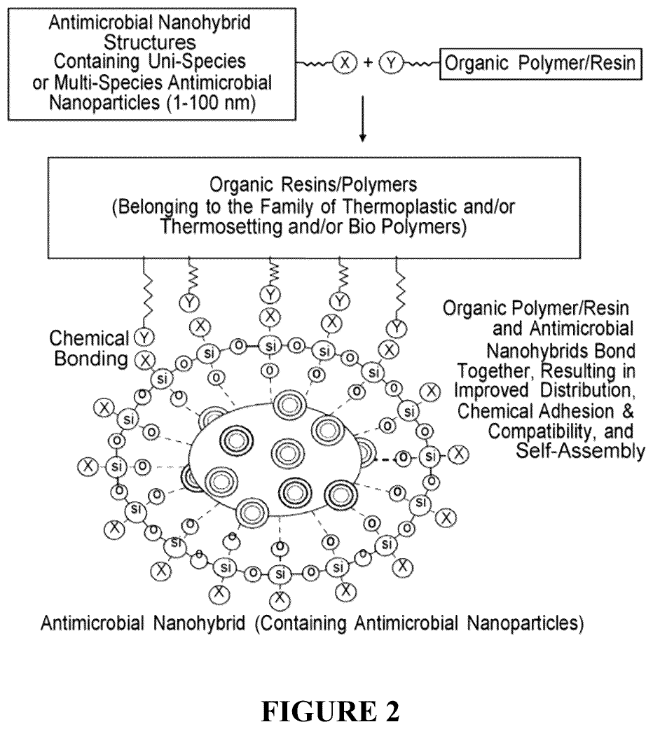

The reaction scheme can include the hydrolysis of the three hydrolyzable groups (Y) of organosilanes. This is followed by condensation to from oligomers and their hydrogen bonding with the surface hydroxyls of antimicrobial nanohybrids. Finally, as reaction concludes with curing, covalent linkages are formed between silicon of organosilane and antimicrobial nanohybrid surface. The organofunctional group (X) remain available for further reaction and bonding. Such reactive organofunctional groups (especially hydrophobic/nonpolar surface treatment) can chemically bond with organic materials, and hence, facilitates the organofunctionalized nanohybrids to covalently bond with polymers as depicted in FIG. 2. The nanohybrid compositions and/or the nanocomposite compositions can include, but are not limited to: synthetic materials with organic and inorganic components that are bonded or linked together by covalent bonding or noncovalent bonding (e.g., hydrogen bond, van der Waals force or electrostatic force) at nanometer scale.

The silane functionalization of the nanohybrid structures can include, but are not limited to: one or more reactive mixing treatments, one or more anhydrous liquid phase depositions, one or more vapor phase depositions, and combination thereof. The reactive mixing treatment can include, but is not limited to: mixing an appropriate organosilane in the form of a concentrate (typically, 0.5-1.0 wt % of nanohybrid weight) or a hydrolyzed solution (typically 0.5-2.0 wt %) with nanohybrid structures (in dry condition or wet state in the presence of a compatible a solvent solution) at room temperature. This is followed by filtering out and/or heat assisted dry curing (˜100-150° C.) of the excess solution to yield organofunctionalized nanohybrid structures. Reactive mixing treatment can simultaneously execute the necessary steps of hydrolysis, condensation, hydrogen bonding, and covalent bonding to yield organofunctionalized antimicrobial nanohybrid structures.

In an embodiment, processing techniques can facilitate the dispersion and bonding of antimicrobial nanohybrid structures within the polymer matrix, including covalent bonding between organofunctional groups and polymer networks and forming/shaping polymeric parts/products with desired configuration and antimicrobial properties for industrial and consumer use. In an embodiment, the covalent bonding between a thermoset urethane polymer and amino functionalized nanohybrid structure during polymer processing. Surfactants can be added to the nanocomposite make a dispersion containing silver nanoparticles.

Polymer compounding or melt blending can include mixing and/or blending polymers/copolymer resins with nanohybrid structures and, optionally, other additives relevant for the polymeric products. The polymer can be processed through different industrially available shaping or forming techniques, including but not limited to: thermoforming, compression and transfer molding, rotational molding and sintering, extrusion and extrusion-based processes, injection molding, blow molding and/or plastic foam molding. All these processes utilize some constraint followed by cooling/curing to form antimicrobial polymer nanohybrids in desired shape and size configurations (such as films, tubes, fibers, sheets, and other configurations).

Polymer solution casting is a processing technique where the antimicrobial nanohybrid structures are thoroughly mixed and dispersed (using powder dispersion, solution mixing and/or wet milling/grinding procedures) in organic polymers dissolved or dispersed in a solution. The mixed solution is coated onto a carrier substrate, and then the water or solvent is removed by drying to create a solid layer on the substrate. The resulting cast layer can be left as an antimicrobial coating overlayer or can be stripped from the carrier substrate to produce a standalone antimicrobial nanohybrid film.

The processing techniques can facilitate the dispersion and bonding of antimicrobial nanohybrid structures within the polymer matrix, including covalent bonding between organofunctional groups and polymer networks and forming/shaping polymeric parts/products with desired configuration and antimicrobial properties for industrial and consumer use. In an embodiment, the covalent bonding between a thermoset urethane polymer and amino functionalized nanohybrid structure during polymer processing.

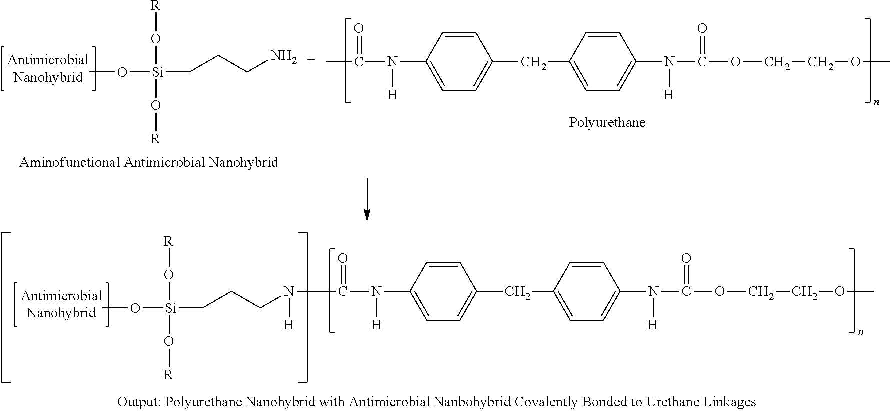

In one or more embodiment, the nanohybrid compositions and/or the nanocomposite compositions can be used in a wide variety of commercial products. For example, the nanohybrid compositions and/or the nanocomposite compositions can be used to make water storage tanks; water filters; film wraps; food packing materials; packing materials; plastic bags; packaging materials, such as for flowers; liquid filters; powders; garbage containers; garbage bags; fabrics; textiles; pillows; pillowcases; sheets; cleaning products; carpets; floor mates; personal protective equipment; medical garments; hospital beds; bedding; medical infusion tubing; medical stents; dental implants; orthopedic implants; urine drain units; and medical furniture. In an embodiment, the nanohybrid compositions and/or the nanocomposite compositions can be used with food grade polyethylene. For example, a powdered nanocomposite composition can be mixed with food grade polyethylene to create a charged pellet that can be used in a variety of products, such as film wraps, plastic bags, TUPPERWARE® by Tupperware Corporation, and other similar food containers. The nanocomposite compositions and/or the nanohybrid compositions can be used to provide antimicrobial, antifungal, anti-odor, or anti-mold properties. The nanocomposite compositions and/or the nanohybrid compositions can at least partially inhibit the growth bacteria, fungi, viruses, and mixtures thereof.

In an embodiment, the physical structures of the commercial product can be at least partially coated with a nanohybrid composition and/or nanocomposite composition by at least partially dipping the physical structure into solution of the nanohybrid composition and/or nanocomposite composition. The coated physical structures can then be at least partially immersed in water to release the antimicrobial metal ions, to provide effective anti-microbial, anti-odor, and anti-stain properties.

In an embodiment, the nanohybrid composition and/or the nanocomposite composition can be used to make filter media for purification and decontamination of above described microbial species from potable and non-potable water. In another embodiment, the nanohybrid compositions can be filled in a filter cartridge, bag, or housing with size and volume configurations depending on the overall quantity and flow rate of water. The nanohybrid compositions and/or the nanocomposite compositions can have high porosity or adsorption spaces. During water treatment and filtration, not only they capture contaminant particles between grains but also adsorb and capture contaminants in their pores. The porosity significantly increases the surface area that facilitates greater contact and interaction of water with the antimicrobial nanoparticles while permeating through the nanohybrid filter media resulting in enhanced microbial inhibition.

The nanocomposite compositions and/or the nanohybrid compositions can provide antimicrobial characteristics to kill and/or inhibit growth and propagation of a broad spectrum of pathogenic and infectious microbes, including but not limited to bacteria, fungi, and viruses. The bacteria can include, but are not limited to: acid producing bacteria, sulphate reducing bacteria, gram-positive, gram-negative bacteria, and mixtures thereof. Examples of such bacteria species can include those in US2013/0108702A1, which is incorporated herein as reference. The microbes can include, but are not limited to: yeasts, rusts, smuts, mildews, molds, and mixtures thereof. The microbes can include, but are not limited to: Aspergillus, Acremonium, Penicillium, Cladosporium, Ophiostoma, Magnaporthe, Fusarium, Mucor, Nerospora, Rhizopus, Tricophyton, Uredinalis, Botryotinia, Phytophthora, Stachybotrys genera, and mixtures thereof. The viruses can include, but are not limited to: rhinoviruses, influenza viruses, human coronavirus, varicella viruses, measles virus, hantavirus, viral meningitis, SARS virus, and mixtures thereof.

The nanohybrid compositions and/or the nanocomposite compositions can be used for microbial water filtration for purification/sterilization of gram-positive and gram-negative bacteria, acid producing bacteria, sulphate reducing bacteria, fungi, and virus species from potable, non-potable, and industrial process water and as well as from the filtration unit and related components. Based on the results of performance testing set forth below, the nanohybrid composition can be capable of superior microbial purification and decontamination of water.

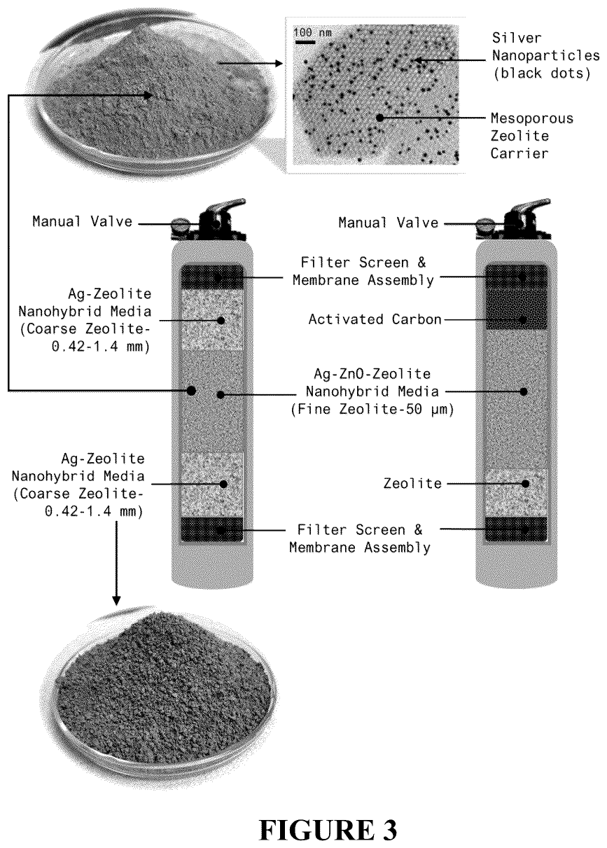

An embodiment of a microbial filtration unit that uses the nanohybrid composition is shown in FIG. 3. The water is permeated through the nanohybrid composition filter media. To prevent leaching during water flow, the nanohybrid filter media is securely confined in microporous and/or nanoporous cages. When water permeates through the nanohybrid filter media, the porosity of carrier materials (e.g., zeolite) combined with hydrophilic/adsorptive organosilane modification forces high surface area interaction or contact of water with the immobilized antimicrobial nanoparticles (e.g., silver nanoparticles). During this interaction, the antimicrobial nanoparticles releases ions (e.g., Ag ions from silver nanoparticles) which not only kill and inhibit the colonialization of the microbes in the filtered water but also the microbes trapped within the filter.

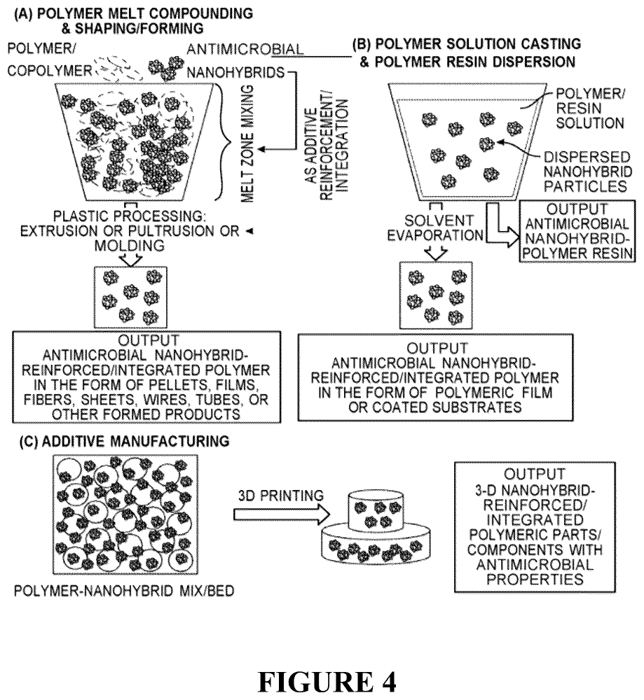

In an embodiment, various plastic and/or polymer processing techniques can used for manufacturing the inventive polymer nanohybrids with the antimicrobial nanohybrid structures depending on the quantity and production rate, dimensional accuracy and surface finish, form and detail of the product, nature of polymeric material and size of final product. The incorporation of the antimicrobial nanohybrids in polymers to form polymer nanohybrids can be accomplished by the following processing techniques as shown in FIG. 4. Polymer compounding or melt blending, shaping, or forming, polymer solution casting, and additive manufacturing.

In an embodiment, a method for making nanocomposites can include depositing silver in the nanometer size range on a low cost, inert, and environmentally safe non-metallic carrier in nano to micron size range and then integrate it in polymeric structure as antimicrobial, anti-fungal, anti-odor, and anti-stain agent are provided. Silver nanoparticles have high surface to volume ratio, allowing an extremely low concentration to be highly effective. The methods can allow for easier mixing and dispersion in industrial processes as well as lower overall material cost, while maintaining excellent antimicrobial efficacy.

In an embodiment, a method for making water storage containers with antimicrobial and antifungal functions is to produce the composite with metallic nanoparticles. The synthesis of such composites is based on a hydrothermal process. In this process, non-metallic particles of nano and micrometer sizes are mixed with silver nitrate, water, and reducing agent, for example, hydrazine, to deposit silver nanoparticles on the surfaces of the non-metallic particles.

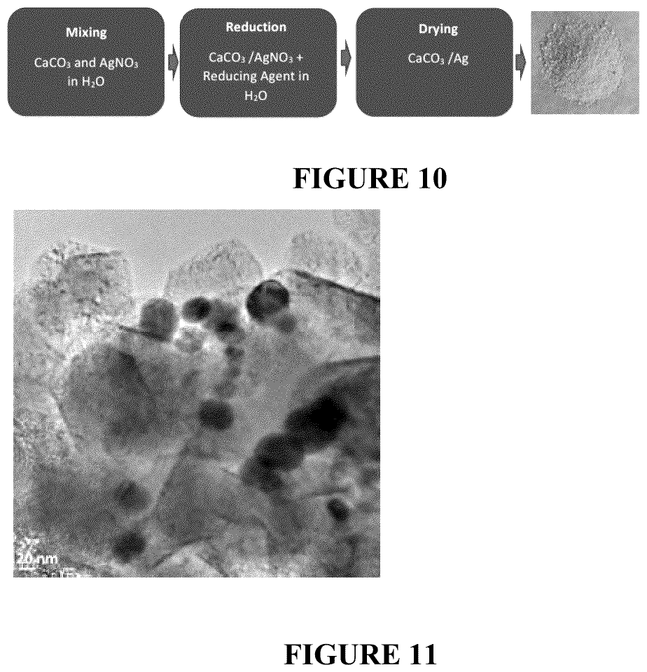

FIG. 10 shows embodiment of a production process that uses the nanohybrid compositions and/or the nanocomposite compositions. The process utilizes commercial grade non-metallic powders as the primary raw material. Based on experiments, a variety of materials are suitable as carrier. Out of the materials tested, calcium-based material is identified as a favorable material for this invention as it is widely used as filler for polymeric material. As shown in FIG. 10, the powder production process includes three primary production phases—mixing, reduction, and drying. During the mixing phase, non-reacting materials are combined and mixed. These materials can be the non-metallic, silver compound, and water or a combination of non-metallic compounds, silver compound, one or more additives, and water. The material mixed during this step has the consistency of a paste and is ready for reduction. During reduction, a reducing agent is added to the paste and mixed. During this step, the silver compound is reduced to metallic phase, which deposits primarily on the non-metallic particles. A subsequent heating of the mixture at 40-165° C. under saturated steam environment completes the production phase. Next, the product is dried in air, vacuum oven, or by other means. The resulting product can be pulverized into a powder.

In an embodiment, a method for using the nanohybrid compositions and/or the nanocomposite compositions can include, at least partially coating fabrics with the nanohybrid compositions and/or the nanocomposite compositions. For example, the nanohybrid compositions and/or the nanocomposite compositions can dispersed in a urethane binder and least partially applying to fabrics. In another embodiment, the method for using the nanohybrid compositions and/or the nanocomposite compositions can include incorporating into textiles.

The nanocomposite can be integrated into polymeric structure to provide bacteria, fungal, stain, and odor resistance. Fabrics coated with the nanocomposites dispersed in a urethane binder can reduce and/or eliminate odor causing bacteria and other bacteria that develop on the vests as they are worn in service.

In an embodiment, the nanocomposite can be mixed with one or more binders and applied as a coating on the interior surfaces of a water storage container. The coating, with a coating thickness in range of about 0.001 to about 500 μm, can release silver ions that provide anti-microbial, anti-odor, and anti-stain function.

The nanohybrid compositions and/or the nanocomposite compositions can be included in many commercial products. For example, the nanocomposite composition can be used in water storage tank, powdered materials or structural polymeric composites, sheets, coatings, and films for antimicrobial surfacing, water storage, water transportation, and filtration.

The one or more membranes and/or filters can include, but are not limited to: fiber membranes and filters, poly pads, mechanical filter media, filter media roll, porous filter pads, foam filter, biofilter, filter bags, surface films, membrane cartridge, filter vessel, capsule filters, and porous supports.

During microfiltration, ultrafiltration, nanofiltration, and/or reverse osmosis filtration with such nanohybrid polymer-based membranes and filters, the antimicrobial nanoparticles will assist in long-term microbial sterilization of filters and water purification. In addition to antimicrobial properties, the incorporation of antimicrobial nanohybrids structures can also strengthen/reinforce the polymer matrix by introducing unique properties, such as mechanical strength, toughness and electrical or thermal controlled properties. For the inventive polymer nanohybrids, hydrophobic (non-polar) organofunctional nanohybrid structures are preferred for covalent bonding and enhanced compatibility with nonpolar organic polymers (thermoplastics and thermosets).

The nanohybrid compositions and/or the nanocomposite compositions can be used for microbial purification and decontamination of water as primary media in a well-designed filtration cartridge/system or as secondary media in conjunction with other filter media/purifiers, such as activated carbon, alumina, mixed media, or urea formaldehyde membranes. During water treatment and filtration, the nanohybrid compositions and/or the nanocomposite compositions filter media traps bacteria and microbes which are killed by the antimicrobial nanoparticles, and at the same time, releases antimicrobial ions which facilitates water to inhibit the growth of microorganisms and their biofilms over time.

In one or more embodiments, the nanohybrid compositions can include, but are not limited to: selective integration and immobilization of inorganic antimicrobial nanoparticles with other inorganic and organic materials and, then using them as treatment or filter media for microbial purification and sterilization of water by killing, inhibiting, and/or reducing the growth/colonization of bacteria and other pathogenic/contaminating microorganisms. The nanohybrid compositions also relate to forming additional/secondary level of water treatment to reduce the growth of microbial biofilms within the filter. The nanohybrid compositions can be used for microbial water purification and sterilization by themselves or in conjunction with other commercially available filter/treatment media and filtration systems. In an embodiment, filtering can include, but is not limited to: purification and/or decontamination (disinfection and sterilization) of microbial species (bacteria, fungi, and viruses) from potable and non-potable water.

The nanocomposite compositions and/or the nanohybrid compositions can be integrated into the respective polymers for co-extrusion process to make the interior layer for water storage tanks. An interior layer of the water storage tank is supported by other structural layers to provide the rigidity requirement for a water storage tank. In such a physical form, the interior layer is anticipated to provide similar long-lasting anti-microbial, anti-odor, and anti-stain functions as aforesaid structural composites because it can release silver ions effectively as soon as it is wetted with water.

FIG. 11 shows a transmission electron microscopy (TEM) micrograph of the nanocomposite composition powder. Discrete silver nanoparticles can be observed on the surface of calcium based nano- and micro-particles. The powder was then mixed with a binder, a dispersion agent and sufficient amount of water to form the anti-microbial and anti-odor coating. The effectiveness of the antimicrobial and anti-odor particles in polyurethane as coating on a fabric was investigated. Typically, antimicrobial tests are performed in accordance with AATCC 100, Assessment of Antibacterial Finishes on Textile Materials, at the Antimicrobial Test Laboratories, LLC, Round Rock, Texas.

TABLE 1
Antimicrobial efficacy testing results based on E. coli, a major health hazard for drinking water

| | | | | | Equivalent | Tested | |
|---|---|---|---|---|---|---|---|
| | Wt. % of | | Binder | | Dry Powder | Sample | % Reduction |
| Sample | Nano Silver | Carrier | Concentration | Surfactant | on the Fabric | Size | vs. Control |
| ID | on Carrier | Material | (wt %) | (SDS, %) | (g) | (in²) | (E. coli) |
| 1 | 1.0 | CaCO₃ | 20 | No | 0.20 | 2.0 × 2.0 | 96.78 |
| 2 | 1.0 | CaCO₃ | 20 | Yes | 0.20 | 2.0 × 2.0 | 99.99985 |

Table 1 shows the results of antimicrobial testing for samples. Clearly, fabric samples with nano-silver coating demonstrated positive results for E. coli, a major bacteria for drinking water, tested for coating with silver particles percentage of 1.0%, equivalent to 0.2 gram of the dry nanocomposite powder. This indicated that reducing the usage of nano-silver in the coating while still maintaining higher antimicrobial efficacy in reference to commercially available anti-microbial products is practical.

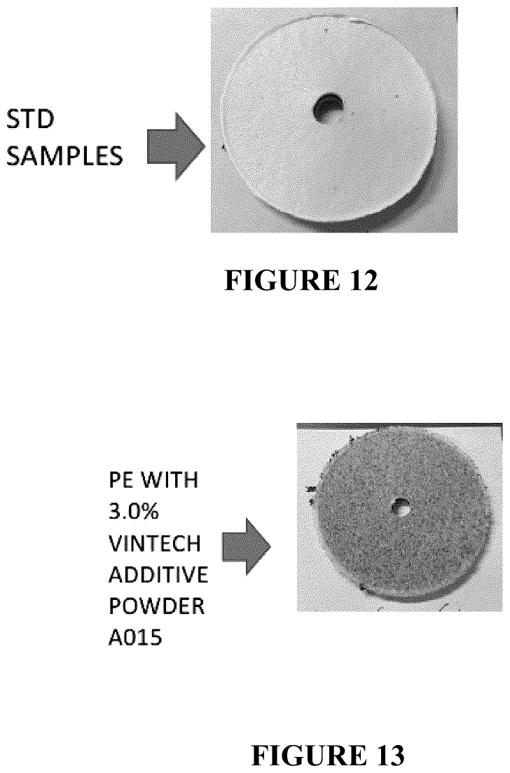

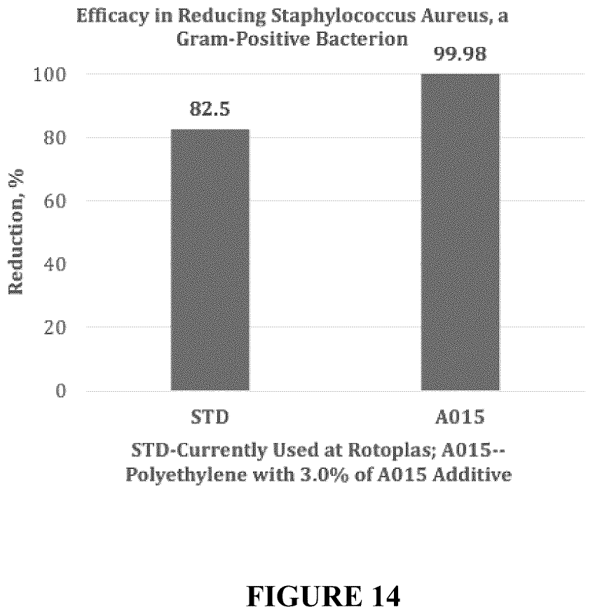

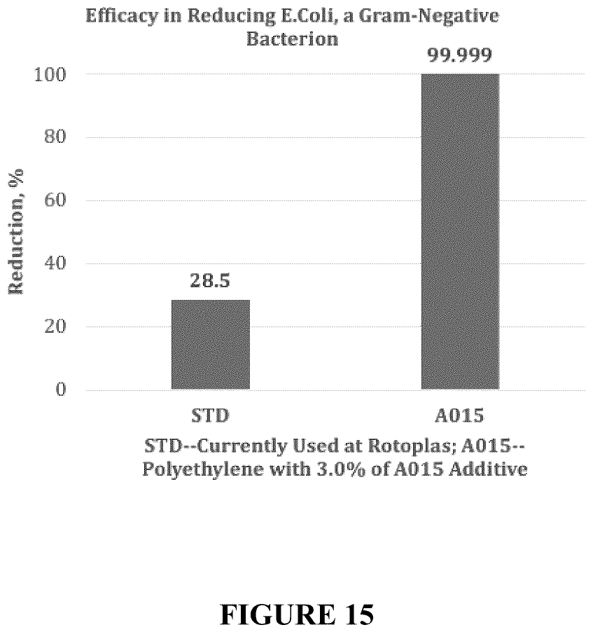

FIGS. 12-15 show the results of additional testing of another embodiment of the nanocomposite. Specifically, the figures show the results of antimicrobial efficacy testing of a plastic composite materials, specifically, inhibiting gram-negative organism, E. coli, and gram-positive organism, S. aureus by following ISO 22196:2011. Results shown are the average of three tests.

As shown circular discs were used having a diameter of 53-56 millimeters and a thickness of 4-8 millimeters. Standard Rotoplas materials were used to create the control (or STD) discs. A 3% additive powder of compound A015 was mixed with polyethylene to create the testing discs. Compound A015 comprises 1.5% nanosilver and 98.5% calcium carbonate. As shown, the inclusion of only 3% of the A015 compound greatly increases the efficacy in reducing both S. aureus and E. coli growth, with a greater than 70% increase in the efficacy against E. coli.

### EXAMPLES

To provide a better understanding of the foregoing discussion, the following non-limiting examples are offered. Although the examples can be directed to specific embodiments, they are not to be viewed as limiting the invention in any specific respect. All percentages and parts are based on weight unless otherwise indicated.

### Example 1: Ag-Zeolite and Ag—CaCO₃—ZnO Nanohybrid Structures

Ag-Zeolite and Ag—CaCO₃—ZnO nanohybrid structures derived from chemical reduction and organofunctionalization. In Example 1, Ag-clinoptilolite Zeolite and Ag—CaCO₃—ZnO structures were first manufactured via chemical reduction method, as listed in Table 1.

TABLE 1
Ag-Clinoptilolite Zeolite and Ag—CaCO₃—ZnO structures

| Composition | 010-101 | 010-102 | 010-702 | 010-703 |
|---|---|---|---|---|
| Granulated Particles 1: | 0 | 0 | 89 | 89 |
| Inorganic CaCO₃ (wt. %) | | | | |
| APS: ~150 nm | | | | |
| Granulated Particles 2: | 0 | 0 | 10 | 10 |
| Inorganic ZnO (wt. %) | | | | |
| APS: ~100 nm | | | | |
| Granulated Particles 3: | 95 | 0 | 0 | 0 |
| Clinoptilolite Zeolite (wt. %) | | | | |
| APS: 50 μm | | | | |
| Granulated Particles 4: | 0 | 95 | 0 | 0 |
| Clinoptilolite Zeolite (wt. %) | | | | |
| APS: 0.4-1.4 mm | | | | |
| Metal salts/Nanoparticle | 8 | 8 | 1.6 | 1.6 |
| Precursor (g): Silver Nitrate | | | | |
| (wt. %) (AgNO₃) | | | | |
| Reducing agent (wt. %) | 5 | 5 | 1 | 1 |
| Surfactant (wt. %) | 1 | 1 | 0 | 2.5 |
| Output Composition | 5%-95% | 5%-95% | 1%-89%-10% | 1%-89%-10% |
| | Ag-Zeolite | Ag-Zeolite | Ag—CaCO₃—ZnO | Ag—CaCO₃—ZnO |

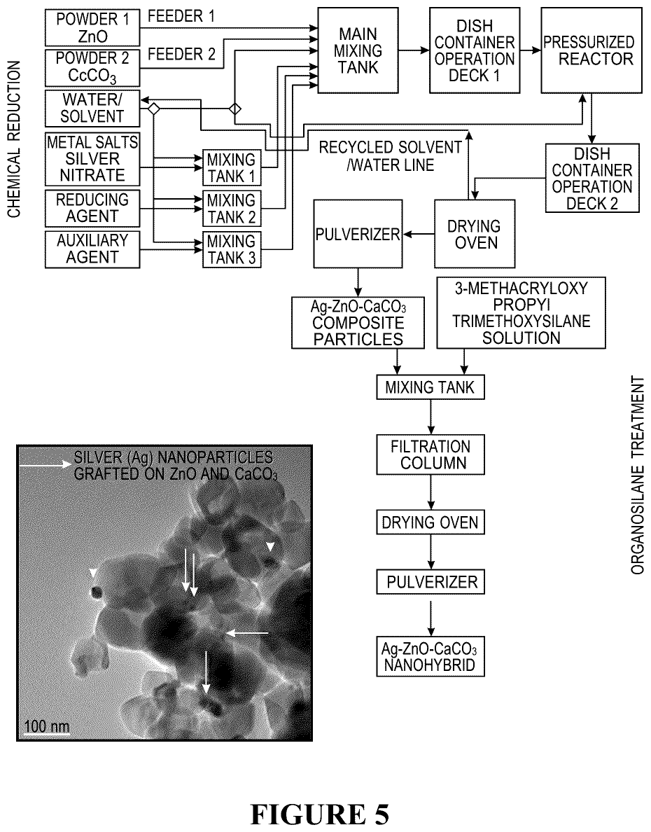

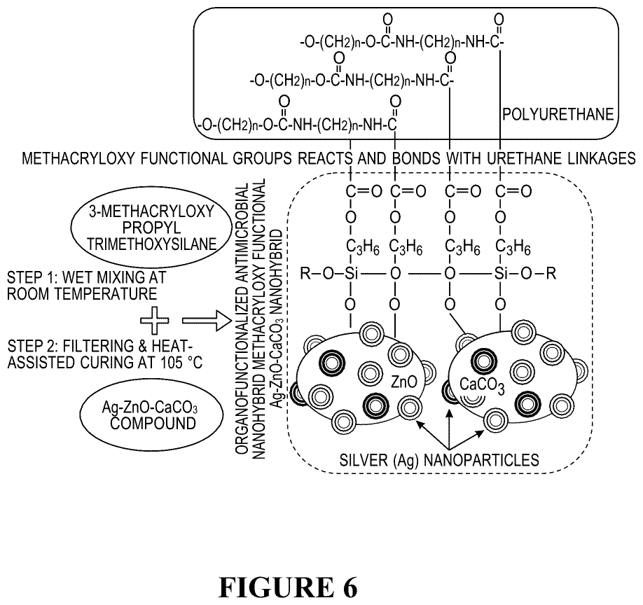

As shown in FIG. 4, the process involved saturating the inorganic carrier materials with silver nitrate (as metallic salt precursor) in an aqueous alcohol solution. For the above compositions, commercially available clinoptilolite zeolite, ZnO and CaCO₃ particulates were used. The saturated aqueous mixture was then reacted with hydrazine monohydrate as reducing agent for reduction of Silver Nitrate to Silver (Ag) nanoparticles deposited in the matrix of zeolite (010-101 and 010-102) and ZnO—CaCO₃ (010-701 and 010-703). As shown in the TEM image of the nanohybrid structure in FIG. 5, spherical-shaped silver nanoparticles (˜15-20 nm) can be seen deposited on the inorganic carrier particles. As mentioned in the description of invention, the metallic salts (nanoparticles precursors) can be any hydrolyzable or water-soluble metal salts which can be reduced to desired species of metallic nanoparticles with antimicrobial properties.

As next step of nanohybrid manufacturing, the 010-101 and 010-102 Ag-zeolite based compositions (as obtained from chemical reduction step) were organofunctionalized using a commercially available N-(3-triethoxysilylpropyl) gluconamide to generate hydrophilic nanohybrid filter media. N-(3-triethoxysilylpropyl) gluconamide is a water-soluble hydroxy functional trialkoxy silane with hydrophilic properties. 010-702, and 010-703 compositions were organofunctionalized using a commercially available 3-methacryloxypropyl trimethoxysilane to generate methacryloxy functionalized nanohybrid structures, as depicted in FIGS. 5 andx 6. 3-Methacryloxypropyl trimethoxysilane is a di-functional organosilane having a reactive non-polar acrylic group (to bond with nonpolar polymers) and three hydrolyzable methoxy groups (to bond with the nanohybrids), thereby acting as an interphase bridge to bond antimicrobial nanohybrids with organic polymers as shown below.

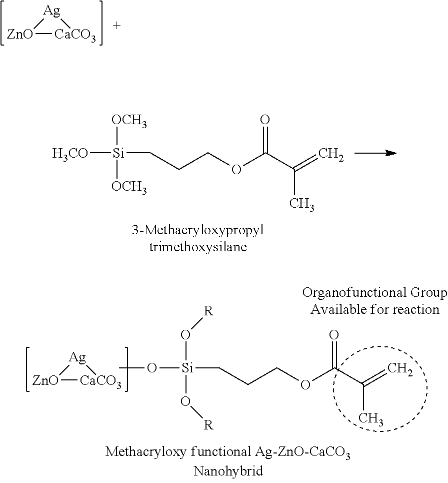

As example description, to obtain methacryloxy functionalized nanohybrid structures, the compositions obtained from chemical reduction step were mixed with a 2.0 wt. % solution of 3-Methacryloxypropyl trimethoxysilane in a 50-50 mix of DI water and methanol. This was followed by filtering out the excess solvent and curing the mixture at 105° C. until it completely dried. The dried compound was then pulverized in a low-powered hammer mill to obtain fine particulates of organofunctionalized nanohybrid structures. Although a different solvent system and curing temperature was adopted, a similar reaction process sequence was implemented to obtain hydroxy functional Ag-Zeolite nanohybrids using N-(3-Triethoxysilylpropyl) Gluconamide.

### Example 2: Application of Organofunctional Ag-Zeolite Nanohybrid Media (010-101) for Frac Water Treatment

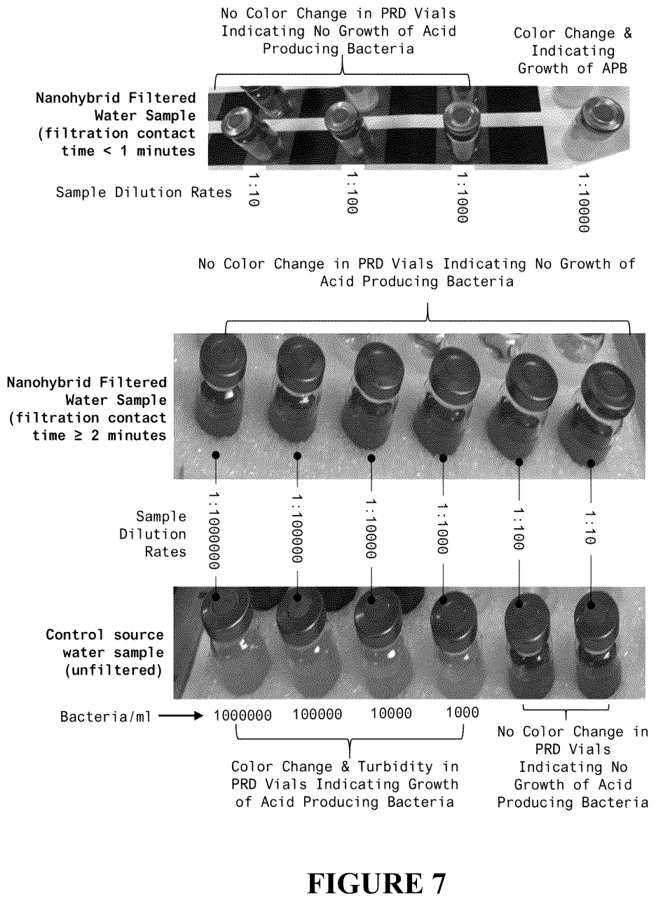

To increase permeability of shale gas and oil formations, millions of gallons of water are injected into wells during hydraulic fracturing. During and post fracturing process, microbial growth and colonization in frac fluids presents a serious concern leading to deleterious microbially induced contamination and corrosion in the wells and equipment. Specially, the growth of acid producing bacteria (APB) lowers the pH and such mildly acidic conditions are sufficiently corrosive to impact the integrity of any metallic structures even in the absence of oxygen. Periodically, significant quantities of toxic biocides are injected to control the microbial growth which have been showed ineffective in killing all the bacteria in frac fluids by many studies. To address this problem, organofunctional Ag-Zeolite based nanohybrid filter media (010-101) was tested for controlling the growth of bacteria in frac water. During testing, source water was passed through a single filter cartridge containing Ag-Zeolite based nanohybrid filter media with variable contact time of <1 minutes and ≥2 minutes between the flowing water and the nanohybrid media. The unfiltered and filtered source water was tested for growth of acid producing bacteria (APB) using phenol red dextrose (PRD) culture media as per NACE standard TM0194-2004. In this test, water sample is inoculated in PRD vials (original color of PRD broth media—red color) at different dilutions—from 1:10 to 1:1000000 for 14 days and change of PRD vial color from red to yellow with turbidity indicates the growth of acid producing bacteria (APB). If APB growth is inhibited, the PRD vials remain red colored. As shown in FIG. 7, the control unfiltered water showed growth of APB—up to 1000000 bacteria/ml of water. Water filtered with nanohybrid media showed no growth of APB up to 1:1000 dilution for <1-minute contact time during filtration. For ≥2-minute contact time, filtered water showed no growth of acid producing bacteria at all dilution levels.

### Example 3: Application of Organofunctional Ag-Zeolite Nanohybrid Media for Microbial Purification and Sterilization of Contaminated Water

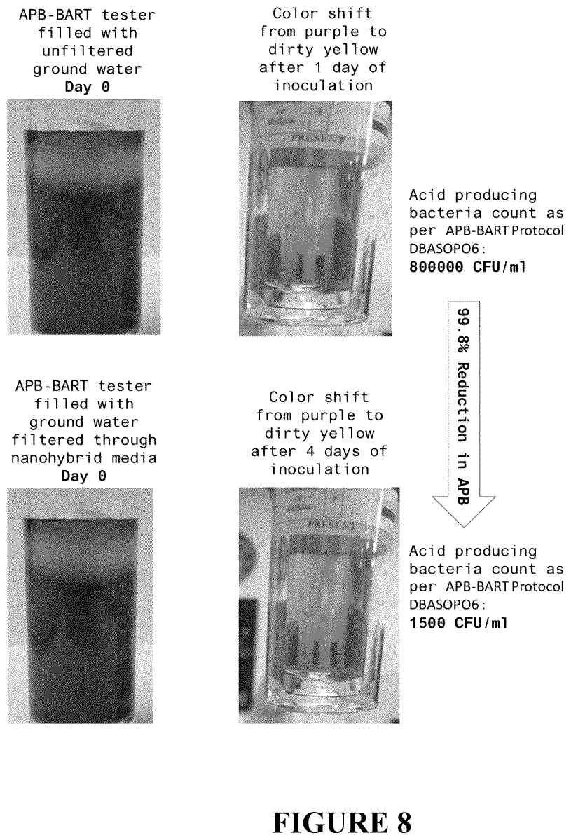

During testing, contaminated ground water with TDS-1200 PPM (unsafe levels of TDS as per EPA) was passed through a filter cartridge (single column cartridge) containing Ag-Zeolite based nanohybrid filter media (010-101 composition) with a contact time of ˜ 1 minutes between the flowing water and the nanohybrid media. APB-BART Protocol DBASOPO6 was implemented to detect the reduction in population of acid producing bacteria (colony forming units-CFU/ml) in contaminated water before and after filtration through the nanohybrid media. As shown in FIG. 8, filtration with nanohybrid media demonstrated excellent antimicrobial efficacy by reducing the population of acid producing bacteria by 99.81% as compared to control (unfiltered) water. With an increase in the nanohybrid media volume and/or multi column filter system, it is very feasible for the inventive nanohybrid filter media to achieve >99.9% reduction of bacterial population.

### Example 4: Nanohybrid Ag—Cu—SiO₂/Polyamide

Ag—Cu—SiO₂ antimicrobial nanohybrid structure was manufactured by via chemical reduction method by reducing and depositing 1.5 wt. % Silver and 1.5 wt. % Copper nanoparticles (5-10 nm) from their salt precursors (Silver nitrate and Copper (II) sulfate) on inorganic silicon dioxide (SiO₂) particles ranging between 25-100 nm in size. The resultant Ag—Cu—SiO₂ composition was then treated with an aminofunctional silane coupling agent (3-Aminopropyltrimethoxysilane). As shown below, the objective of organosilane treatment was to generate organofunctional amine (—NH₂) groups for improved bonding and compatibility of antimicrobial nanohybrids with polyamides (a polymer belonging to the family of thermoplastic polymers).

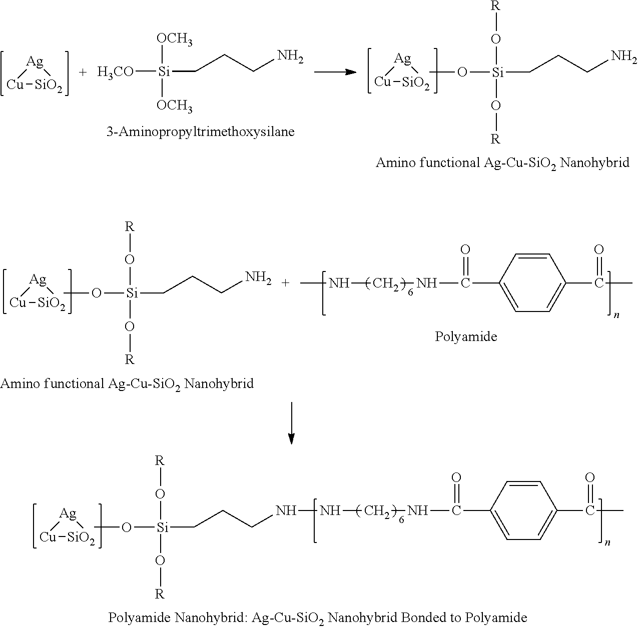

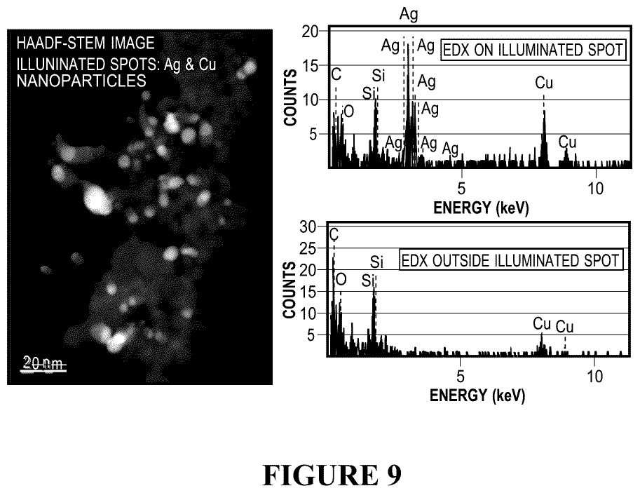

To confirm the deposition of Ag and Cu nanoparticles on inorganic silicon dioxide, high-angle annular dark-field scanning transmission electron microscopy (HAADF-STEM) was performed on the nanohybrid structure as shown in FIG. 9. HAADF-STEM confirmed the deposition of Ag and Cu nanoparticles (as illuminated spots) on inorganic silicon dioxide particles. The location and chemical identity of nanoparticles was further confirmed from energy dispersive X-ray analysis (EDX). As shown in FIG. 8, EDX analysis confirmed elemental composition of silver (from Ag nanoparticles), copper (from Cu nanoparticles) and silicon (from organosilane) on the illuminated spots. While EDX analysis on the non-illuminated spots returned elemental signature of only silicon (Si) derived from organosilane and inorganic silicon dioxide.

The polyamide nanohybrids were manufactured by dispersing the aminofunctional nanohybrid structures (5.0 wt. %) in a solvent-borne polyamide binder and then, forming thin composite films via polymer solution casting method. During this process, the aminofunctional groups form covalent linkages with polyamide to form a strongly bonded network of antimicrobial nanohybrids within the composite polymer films. As shown in Table 2, the polymeric films derived from nanohybrid integrated polyamide yielded excellent antimicrobial performance by inhibiting the colonialization of both gram positive and gram-negative microbes by 99.99%. Such performance is very encouraging to expand the use of such polymer nanohybrids for manufacturing of fibers and their end products such as fibrous filter media and membranes with antimicrobial properties.

TABLE 2
Polymeric Films Derived From Nanohybrid
Compositions Including Polyamide

| | | Contact | | |
|---|---|---|---|---|
| | | Time | | % |
| Microorganism | Sample | (hr.) | CFU/Swatch | Reduction |
| Staphylococcus | Control PA film | 0 | 7.20E+04 | No |
| aureus | (no nanohybrid) | 24 | 1.10E+06 | reduction |
| | Ag—Cu—SiO₂/PA | 0 | 7.95E+04 | 99.9874% |
| | nanocomposite | 24 | <1.00E+01 | |
| Salmonella | Control PA film | 0 | 1.31E+05 | No |
| enterica | (no nanohybrid) | 24 | 2.52E+07 | reduction |
| | Ag—Cu—SiO₂/PA | 0 | 1.28E+05 | 99.9922% |
| | nanocomposite | 24 | <1.00E+01 | |

FIG. 11 shows a transmission electron microscopy (TEM) micrograph of the nanocomposite composition powder. Discrete silver nan particles can be observed on the surface of calcium based nano- and micro-particles. The powder was then mixed with a binder, a dispersion agent and sufficient amount of water to form the anti-microbial and anti-odor coating. The effectiveness of the antimicrobial and anti-odor particles in polyurethane as coating on a fabric was investigated. Typically, antimicrobial tests are performed in accordance with AATCC 100, Assessment of Antibacterial Finishes on Textile Materials, at the Antimicrobial Test Laboratories, LLC, Round Rock, Texas.

Table 3 shows the results of antimicrobial testing for samples. Clearly, fabric samples with nano-silver coating demonstrated positive results for Escherichia coli, a major bacteria for drinking water, tested for coating with silver particles percentage of 1.0%, equivalent to 0.2 gram of the dry nanocomposite powder. This indicated that reducing the usage of nano-silver in the coating while still maintaining higher antimicrobial efficacy in reference to commercially available anti-microbial products is practical.

TABLE 3
Antimicrobial Efficacy testing results based on E. coli

| | | | | | Equivalent | Tested | |
|---|---|---|---|---|---|---|---|
| | Wt. % of | | Binder | | Dry Powder | Sample | % Reduction |
| Sample | Nano Silver | Carrier | Concentration | Surfactant | on the Fabric | Size | vs. Control |
| ID | on Carrier | Material | (wt %) | (SDS, %) | (g) | (in²) | (E. coli) |
| 1 | 1.0 | CaCO₃ | 20 | No | 0.20 | 2.0 × 2.0 | 96.78 |
| 2 | 1.0 | CaCO₃ | 20 | Yes | 0.20 | 2.0 × 2.0 | 99.99985 |

FIGS. 12-15 show the results of additional testing of another embodiment of the nanocomposite. Specifically, the figures show the results of antimicrobial efficacy testing of a plastic composite materials, specifically, inhibiting gram-negative organism, E. coli, and gram-positive organism, S. aureus by following ISO 22196:2011. Results shown are the average of three tests. As shown circular discs were used having a diameter of 53-56 millimeters and a thickness of 4-8 millimeters. Standard Rotoplas materials were used to create the control (or STD) discs. A 3% additive powder of compound A015 was mixed with polyethylene to create the testing discs. Compound A015 comprises 1.5% nanosilver and 98.5% calcium carbonate. As shown, the inclusion of only 3% of the A015 compound greatly increases the efficacy in reducing both S. aureus and E. coli growth, with a greater than 70% increase in the efficacy against E. coli.

One of ordinary skill in the art will readily appreciate that alternate but functionally equivalent components, materials, designs, and equipment may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention. Furthermore, all patents, test procedures, and other documents cited in this application are fully incorporated by reference to the extent such disclosure is not inconsistent with this application.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. It should also be appreciated that the numerical limits can be the values from the examples. Certain lower limits, upper limits and ranges appear in at least one claims below. Unless otherwise indicated, all numbers expressing feature sizes, amounts, and physical properties used in the specification and claims are to be understood as being modified in all instances by the term “about.” The term “about” is defined to be +2% of the modified value.

Although the invention has been described with reference to the above examples, it will be understood that modifications and variations are encompassed within the spirit and scope of the invention. Accordingly, the invention is limited only by the following claims.

### REFERENCES

1. Brown, E. D. and G. D. Wright, Antibacterial drug discovery in the resistance era. Nature, 2016. 529(7586): p. 336-343.
2. Theuretzbacher, U., Global antibacterial resistance: The never-ending story. Journal of global antimicrobial resistance, 2013. 1(2): p. 63-69.
3. World-Health-Organisation, (Ed: W. H. Organisation), Geneva 2019.
4. Brandelli, Adriano & Ritter, Ana & Veras, Flávio. (2017). Antimicrobial Activities of Metal Nanoparticles. 10.1007/978-3-319-63790-715.
5. Joshi, Dirgha & Adhikari, Nisha. (2019). An Overview on Common Organic Solvents and Their Toxicity. Journal of Pharmaceutical Research International, 1-18.
6. U.S. Patent Publication No. US2013/0108702A1

What is claimed is:

1. A method for making a nanohybrid composition, wherein the method comprises:
contacting a carrier material with metallic salt precursors to make a first mixture,
contacting the first mixture with a reducing agent reacting and a surface activated agent to make a second mixture, wherein the metallic salt precursors undergo chemical reduction to make metallic nanoparticles, and wherein the metallic nanoparticles are deposited onto the carrier material,
contacting the second mixture with an organosilane coupling agent to make a nanohybrid composition.

2. The method of claim 1, wherein the nanohybrid composition has an antimicrobial efficacy against acid producing bacteria, sulfate reducing bacteria, gram-positive and gram-negative bacteria, fungi, and viruses is greater than 90%.

3. The method of claim 1, wherein the metallic salt precursor is a material selected from the group consisting of: silver, copper, copper oxide, zinc oxide, nickel, selenium, titanium, and titanium dioxide.

4. The method of claim 1, wherein the coupling agent is an organosilane with an organofunctional group.

5. The method of claim 4, wherein the coupling agent is an organosilane with an organofunctional group selected from the group consisting of: a vinyl group, epoxy group, mercapto groups, amino groups, methacryloxy group, isocyanate groups, thiol groups, methoxy groups, hydroxy groups, glycol functional and ethoxy groups.

6. The method of claim 1, wherein the carrier material is selected from the group consisting of granular clinoptilolite zeolite, chabazite, activated carbon, activated alumina, manganese dioxide, anthracite, birm, calcite, magnesium oxide, silica sand, diatomite, zinc oxide, titanium dioxide, graphene, graphene oxide, garnet, chitosan, starch, lignin, nano-fibrillated cellulose, and organic polymers.

7. A method for making a nanohybrid composition, wherein the method comprises:
contacting nanoparticles with a carrier material to make a first mixture,
homogenizing the first mixture utilizing mechanochemical synthesis to make a homogeneous second mixture, and
contacting the second mixture with an organosilane coupling agent to make a nanohybrid composition.

8. The method of claim 7, wherein the nanohybrid composition has an efficacy against acid producing bacteria, sulfate reducing bacteria, gram-positive and gram-negative bacteria, fungi, and viruses is greater than 90%.

9. The method of claim 7, wherein the nanoparticles is selected from the group comprising:
silver, copper, copper oxide, zinc oxide, nickel, selenium, titanium, and titanium dioxide.

10. The method of claim 7, wherein the carrier material is selected from the group consisting of granular clinoptilolite zeolite, chabazite, activated carbon, activated alumina, manganese dioxide, anthracite, birm, calcite, magnesium oxide, silica sand, diatomite, zinc oxide, titanium dioxide, graphene, graphene oxide, garnet, chitosan, starch, lignin, nano-fibrillated cellulose, and organic polymers.

11. The method of claim 7, wherein the coupling agent is an organosilane with an organofunctional group.

12. The method of claim 11, wherein the coupling agent is an organosilane with an organofunctional group selected from the group consisting of a vinyl group, epoxy group, mercapto groups, amino groups, methacryloxy group, isocyanate groups, thiol groups, methoxy groups, hydroxy groups, glycol functional and ethoxy groups.

13. A method for making a nanocomposite composition, wherein the method comprises:
contacting a non-metallic nanoparticle and a metal nanoparticle to make a nanocomposite particle, wherein the metallic nanoparticle is selected from the group comprising: silver, copper, zinc, selenium, and titanium, wherein non-metallic particles are selected from group consisting of: calcium carbonate and silicon dioxide, wherein the metallic nanoparticles have an average particle size in the range from 0.005 to 1 micron, and wherein the non-metallic particles have an average particle size in the range from 0.01 to 40 microns; and
contacting the nanocomposite particle with a polymer to make a nanocomposite composition.

14. The method of claim 13, wherein the polymers are selected from the group consisting of: high-density polyethylene, low-density polyethylene, linear low-density polyethylene, polypropylene, acrylic, polyamide, polycarbonate, polystyrene, acrylonitrile butadiene styrene, polyvinyl chloride, teflon, polyester, polyacrylic acid, polybutylene terephthalate, epoxy, phenolic, vinyl ester, polyurethane, fluoropolymers, cyanate ester, poly ester, urea formaldehyde, silicone/polysiloxane, isoprene polymers, natural polyphenolic polymers, cellulose/nano cellulose, lignin, melanin, cellulose acetate, polyvinylidene fluoride, polysulfone, polyacrylonitrile, polyethersulfone, polyethylene glycol, polyvinyl alcohol, poly(methyl methacrylates), polyacryletherketone, polyethylenimine, polyaniline nanoparticles, aliphatic polyamide, aromatic polyamide, polyethersulfone amide, styrene acrylonitrile, polyether ether ketone, and complex polymers of long-chain fatty acids.

15. The method of claim 13, wherein the nanocomposite composition is a solid.

16. The method of claim 13, wherein the nanocomposite composition is a liquid.

17. The method of claim 13, wherein the antimicrobial efficacy against acid producing bacteria, sulfate reducing bacteria, gram-positive and gram-negative bacteria, fungi, and viruses is greater than 90%.

18. The method of claim 13, wherein the nanocomposite composition is applied to a filter or treatment medium suitable for purification treatment, filtration, and/or decontamination (disinfection and sterilization) of potable, non-potable, or industrial process water from at least one of the following: acid producing bacteria, sulfate reducing bacteria, gram-positive bacteria, gram-negative bacteria, fungi, or viruses.

19. The method of claim 13, wherein said structures are incorporated into a filter for neutralizing at least one of the following: acid producing bacteria, sulfate reducing bacteria, gram-positive and gram-negative bacteria, fungi, and viruses trapped within the filter.

20. The method of claim 13, wherein said nanocomposite compositions are used in manufacturing polymer-based membranes, filters, fibrous structures or porous support used in microfiltration, ultrafiltration, nanofiltration, and reverse osmosis filtration of potable, non-potable water, and industrial process water.
